(19) United States
(12) Patent Application Publication
(10) Pub. No.: US 2025/0009649 A1
(43) Pub. Date: Jan. 9, 2025

(54) Implants for Loss of Taste and Smell

(71) Applicant: Cyrano Therapeutics, Inc., Delray Beach, FL (US)

(72) Inventors: Richard GEOFFRION, Delray Beach, FL (US); Ronald KUPPERSMITH, College Station, TX (US)

(21) Appl. No.: 18/741,915

(22) Filed: Jun. 13, 2024

Related U.S. Application Data
(63) Continuation of application No. PCT/US2022/081622, filed on Dec. 15, 2022.
(60) Provisional application No. 63/292,027, filed on Dec. 21, 2021.

(51) Int. Cl. A61K 9/00; A61P 27/00

(52) U.S. Cl. CPC A61K 9/0043; A61P 27/00

(57) ABSTRACT

Disclosed herein are methods of treating chemosensory dysfunction in a subject, comprising administering to the subject a device such as a stent comprising a phosphodiesterase inhibitor. Also disclosed herein are devices comprising phosphodiesterase inhibitors, and kits comprising devices comprising phosphodiesterase inhibitors.

### CROSS-REFERENCE

This application is a continuation of PCT/US2022/081622, filed Dec. 15, 2022, which claims the benefit of U.S. Provisional Application No. 63/292,027, filed Dec. 21, 2021, the disclosure of which is incorporated herein by reference in its entirety.

### SUMMARY

This disclosure relates to methods and devices for treating or preventing chemosensory dysfunction (e.g., anosmia or dysgeusia).

In one aspect, this disclosure provides a method of treating or preventing a chemosensory dysfunction in a subject the method comprising: delivering a stent to a nasal or sinus cavity to treat or prevent the chemosensory dysfunction in the subject, wherein the nasal or the sinus cavity comprises the nasal cavity which includes: the inferior meatus, the middle meatus, the superior meatus, the sphenoethmoid recess, the olfactory cleft; an ethmoid sinus cavity, a maxillary sinus cavity, a frontal sinus cavity, or a sphenoid sinus cavity, wherein the stent: (a) is at least partially impregnated with a phosphodiesterase (PDE) inhibitor or a salt thereof; (b) comprises at least one coating layer on a least a portion of the stent, the coating layer comprising the PDE inhibitor or the salt thereof; or (c) both (a) and (b), wherein the stent is configured to release the PDE inhibitor or the salt thereof, and wherein the PDE inhibitor or the salt thereof comprises theophylline or a salt thereof, cilostazol or a salt thereof, or roflumilast or a salt thereof, or any combination thereof. In some embodiments, a stent can be place at a sinus ostium or the nasal cavity, or the olfactory cleft. In some embodiments, a PDE inhibitor or the salt thereof can comprise theophylline or a salt thereof. In other embodiments, a PDE inhibitor or the salt thereof can comprise cilostazol or a salt thereof. In some embodiments, the coating layer can include a release rate modifier. In some embodiments, a release rate modifier can comprise a polyethylene glycol (PEG). In some embodiments, a release rate modifier can comprise a bovine serum an albumin, a human serum albumin, a collagen, a gelatin, a polylactic acid (PLA), a polyglycolide (PGA), a polylactic-co-glycolic acid (PLGA), a poly(lactide-co-glycolide), a poly(L-lactide-co-ε-ca-prolactone), a polyhydroxybutyrate (PHB), a polyhydroxyvalerate (PHV), a polydioxanone, a polyglactin, polycaprolactone, a polyglyconate, a poly(glycolide-co-trimethylene carbonate), a poly(sebacic acid), a poly(ester urethane), a poly(ester urethane) urea, a PEG, chitosan, or any combination thereof. In some embodiments, the method can further include a second coating layer. In some embodiments, the method can include a plurality of coating layers, for example, at least three coating layers.

In some embodiments, a stent can be a biodegradable stent. In some embodiments, a stent can be a non-biodegradable stent. In some embodiments, a stent can be a bioabsorbable stent. In some embodiments, a stent can be a non-bioabsorbable stent. In some embodiments, a stent can comprise a PHB, a PHV, a polycaprolactone, or any combination thereof. In some embodiments, a stent can be configured to release the PDE inhibitor or the salt thereof over a period of time from about 5 days to about 180 days. In some embodiments, a stent can be configured to release about 500 μg of the PDE inhibitor or salt thereof per day. In some embodiments, a chemosensory dysfunction can comprise ageusia, hypogeusia, dysgeusia, parosmia, phantosmia, anosmia, hyposmia, dysosmia, or any combination thereof. In some embodiments, a chemosensory dysfunction can comprise anosmia, hyposmia, dysosmia, parosmia, or a combination thereof. In some embodiments, a chemosensory dysfunction can comprise smell loss, taste loss, or a combination thereof. In some embodiments, a subject before treatment can have a decreased level of a decreased level of a cyclic nucleotide, a decreased level of a sonic hedgehog, or both, in a nasal mucus sample from the subject in comparison to a reference level associated with normal chemosensory function. For example, in some embodiments, the subject before treatment can have a decreased level of a cyclic nucleotide in a nasal mucus sample from the subject in comparison to a reference level associated with normal chemosensory function, which can be a cyclic nucleotide level of a control population with normal chemosensory function. In some embodiments, the method can further include administering a second therapeutic. In some embodiments, the second therapeutic can be administered concurrently or consecutively. In some embodiments, the method further includes diagnosing the subject with chemosensory dysfunction. In some embodiments, the subject was previously diagnosed with chemosensory dysfunction.

In another aspect, this disclosure provides an implantable stent comprising: a phosphodiesterase (PDE) inhibitor or a salt thereof, wherein the PDE inhibitor or the salt thereof is: (a) impregnated in the stent; (b) comprised in a coating layer on the stent; or (c) both (a) and (b), wherein the stent is configured to release the PDE inhibitor or the salt thereof in a nasal cavity of a subject and wherein the PDE inhibitor or the salt thereof comprises theophylline, a salt thereof, cilostazol, a salt thereof, or any combination thereof.

In a different aspect, this disclosure provides a method of treating or preventing a chemosensory dysfunction in a subject the method comprising: delivering and depositing an implantable device to a nose to treat or prevent the chemosensory dysfunction in the subject, wherein the implantable device: (a) is at least partially impregnated with a phosphodiesterase (PDE) inhibitor or a salt thereof; (b) comprises, on a least a portion of the implantable device, at least one coating layer comprising the PDE inhibitor or the salt thereof; or (c) both (a) and (b), wherein the implantable device is configured to release the PDE inhibitor or the salt thereof, and wherein the PDE inhibitor or the salt thereof comprises theophylline, a salt thereof, cilostazol, a salt thereof or any combination thereof. In some embodiments, a nose can comprise a sinus cavity or a nasal cavity. In some embodiments, a nose can comprise a nasal or sinus cavity and the nasal cavity which includes: the inferior meatus, the middle meatus, the superior meatus, the sphenoethmoid recess, the olfactory cleft and wherein the sinus cavity comprises an ethmoid sinus cavity, a maxillary sinus cavity, a frontal sinus cavity, or a sphenoid sinus cavity. In some embodiments, a nose can comprise a nasal cavity and the nasal cavity can comprise a fossae.

In another aspect, this disclosure provides a method of making an implantable stent comprising: (a) impregnating a stent with a PDE inhibitor or a salt thereof; (b) coating a stent in a layer comprising the PDE inhibitor or the salt thereof; or (c) both (a) and (b), wherein the stent is configured to release the PDE inhibitor or the salt thereof in a nasal cavity of a subject, and wherein the PDE inhibitor or the salt thereof comprises theophylline, a salt thereof, cilostazol, a salt thereof, or any combination thereof.

In a different aspect, this disclosure provides a kit comprising a stent. In some embodiments, the kit can include an implantable stent comprising: a phosphodiesterase (PDE) inhibitor or a salt thereof, wherein the PDE inhibitor or the salt thereof is: (a) impregnated in the stent in at least a portion of the stent; (b) comprised in a coating layer on the stent on at least a portion of the stent; or (c) both (a) and (b), wherein the stent is configured to release the PDE inhibitor or the salt thereof in a nasal cavity of a subject and wherein the PDE inhibitor or the salt thereof comprises theophylline, a salt thereof, cilostazol, a salt thereof, or any combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein can not necessarily be construed to represent a substantial difference over what is generally understood in the art.

As recited by in disclosure, various embodiments may be presented in a range format. It can be understood that the description in range format is merely for convenience and brevity and can not be construed as an inflexible limitation on the scope of the disclosure. Accordingly, the description of a range can be considered to have specifically disclosed all the possible subranges as well as individual numerical values within that range. For example, description of a range such as from 1 to 6 can be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This can apply regardless of the breadth of the range.

The singular forms “a”, “an”, and “the” are used herein and can include plural references unless the context clearly dictates otherwise. Accordingly, unless the contrary is indicated, the numerical parameters set forth in this application are approximations that can vary depending upon the desired properties sought to be obtained.

The terms “determining”, “measuring”, “evaluating”, “assessing,” “assaying,” and “analyzing” are often used interchangeably herein and can refer to forms of measurement and include determining if an element may be present or not (for example, detection). These terms can include quantitative, qualitative or quantitative, and qualitative determinations. Assessing can be alternatively relative or absolute. “Detecting the presence of” includes determining the amount of something present, as well as determining whether it may be present or absent.

The term “substantially” or “essentially” can refer to a qualitative condition that exhibits an entire or nearly total range or degree of a feature or characteristic of interest. In some cases, substantially can refer to at least about: 70%, 75%, 80%, 85%, 90%, 95%, 99%, or 100% of the total range or degree of a feature or characteristic of interest.

The term “at least partially” can refer to a qualitative condition that exhibits a partial range or degree of a feature or characteristic of interest. In some cases, at least partially can refer to at least about: 5%, 10%, 20%, 30%, 40%, 50%, 60%, 70%, 75%, 80%, 85%, 90%, 95%, 99%, or 100% of the total range or degree of a feature or characteristic of interest.

Unless otherwise indicated, open terms for example “contain,” “containing,” “include,” “including,” and the like mean comprising.

As used herein, the term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. The term “substantially” as used herein can refer to a value approaching 100% of a given value. In some cases, the term can refer to an amount that can be at least about 90%, 91%, 92%, 93%, 94%, 95%, 96%, 97%, 98%, 99%, 99.9%, or 99.99% of a total amount. In some cases, the term can refer to an amount that can be about 100% of a total amount.

The term “device,” as used herein can refer to an implantable device. In some cases, a device can comprise an implant. In some cases, a device, such as an implant can comprise a stent. In some cases, a stent can comprise a biodegradable stent or a nondegradable stent. In some cases, a device can have a coating. In some instances, a device can have multiple layers of coatings, for example a device can have 1, 2, 3, 4, 5, 6, 7, 8, 9, or more layers of coatings. In some cases, the coatings can comprise the same or different compositions. A device as used herein can comprise a topical or mucosal (e.g., nasally or oral mucosa) applied patch. In some instances, a patch can comprise a biodegradable patch or a non-degradable patch. In some cases, a patch can comprise a microneedle patch. In some cases, a device can comprise a pump. In some cases, a device can be an active device which can require resupply of a drug. In some cases, a device can be deposited into a cavity. In some cases, a device can be a passive device which may not require the resupply of a drug. In some cases, a device can be an expandable device such as a balloon, which can be inflated before or after implantation. In some cases, a device can have a first compressed configuration enabling low profile delivery, a second expanded configuration for apposition against tissue, and comprise either a single continuous filament or at least two non-intersecting filaments. In some cases, a device herein may be implanted with an implantation device. A device can be a rod, a sphere, a cone, a cube, a rectangle, a square, a circular, a triangle, a polygon, or any shape.

When used herein, a percentage of a compound of a composition can be with respect to a total weight or a total volume of a composition. In some cases, a percentage of a component of a composition can be with respect to a total weight or a total volume of a composition

The terms “administer,” “administering”, “administration,” and the like, as used herein, can refer to methods that can be used to enable delivery of compounds or their salts or compositions to the desired site of biological action. Delivery can include direct application to the affect tissue or region of the body. A composition provided herein can be administered by any method. A method of administration can be by inhalation, intraarterial injection, intracerebroventricular injection, intracisternal injection, intramuscular injection, intraorbital injection, intraparenchymal injection, intraperitoneal injection, intraspinal injection, intrathecal injection, intravenous injection, intraventricular injection, stereotactic injection, subcutaneous injection, or any combination thereof. Delivery can include parenteral administration (including intravenous, subcutaneous, intrathecal, intraperitoneal, intramuscular, intravascular or infusion), oral administration, nasal administration, inhalation administration, intraduodenal administration, rectal administration. Delivery can include topical administration such as a patch to an external surface of a surface. In some instances, an external surface can comprise skin or a mucosal surface. In some cases, delivery can comprise delivery by a device, such as an implantable stent. In some cases, a device (e.g., a stent) can be delivered by a delivery device or a delivery tool, such as, a pair of surgical pliers or forceps. In some case, a device can be delivered and deposited into a nasal cavity, a sinus cavity or both for a period of time. In some instances, a subject can administer the compound, salt thereof, or the stent comprising the compound in the absence of supervision. In some instances, a subject can administer the composition or stent under the supervision of a medical professional (e.g., a physician, nurse, physician's assistant, orderly, hospice worker, etc.). In some cases, a medical professional can administer the composition or stent. In some cases, a cosmetic professional can administer the composition.

As used herein, “treating” of chemosensory dysfunction can include one or more of preventing or reducing the frequency or severity of one or more symptoms, elimination of one or more symptoms or their underlying cause, or improvement or remediation of damage. For example, treatment of chemosensory dysfunction can include, for example, increasing smell acuity and taste acuity from a patient suffering from a viral infection, such as a patient with COVID-19 or influenza and/or causing the regression or disappearance of chemosensory dysfunction.

A “therapeutically effective amount” can refer to an amount of a compound or its salt with or without additional agents that is effective to achieve its intended purpose. Individual patient needs may vary. Generally, the dosage required to provide an effective amount of the compound, salt thereof, or composition containing one or both of these, and which can be adjusted by one of ordinary skill in the art, will vary, depending on the age, health, physical condition, sex, weight, extent of the dysfunction of the recipient, frequency of treatment and the nature and scope of the dysfunction.

The term “subject,” “host,” “individual,” and “patient” are as used interchangeably herein and can refer to animals, typically mammalian animals. Any suitable mammal can be administered a compound, salt, or a composition as described herein or treated by a method as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). Mammals can be any age or at any stage of development, for example a mammal can be neonatal, infant, adolescent, adult or in utero. In some embodiments a mammal is a human. Humans can be more than about: 1, 2, 5, 10, 20, 30, 40, 50, 60, 65, 70, 75, 80, 85, 90, 95, 100, 105, 110, 115 or about 120 years of age. Humans can be less than about: 1, 2, 5, 10, 20, 30, 40, 50, 60, 65, 70, 75, 80, 85, 90, 95, 100, 105, 110, 115 or about 120 years of age. In some cases, human can be from about 1 month to about 12 months old, from about 1 year to about 20 years, from about 15 years to about 50 years, from about 40 years to about 80 years, or from about 60 years to about 110 years. In some cases, a human can be less than about 18 years of age. In some cases, a human can be more than about 18 years of age. A mammal such as a human can be male or female. In some embodiments, a subject can have or can be suspected of having a disease or condition. The subject can be a patient, such as a patient being treated for a condition or a disease, such as a heart disease, hypertension, atrial fibrillation, stroke, renal failure, liver disease, cancer, diabetes, respiratory disease, asthma, chronic obstructive pulmonary disease, bronchitis, emphysema, lung cancer, cystic fibrosis, Coronavirus infection, a viral infection, a bacterial infection, a fungal infection, a parasitic infection, an influenza infection, pneumonia, pleural effusion, or any combination thereof. A subject can be predisposed to a risk of developing a condition or a disease such as a respiratory disease. A subject can be in remission from a condition or a disease, such as a cancer patient. In some instances, a subject can be healthy.

As disclosed herein, the term “phosphodiesterase (PDE) inhibitor” can refer to a compound that can at least partially inhibit the function of a phosphodiesterase (PDE) polypeptide, such as a PDE1, PDE2, PDE3, PDE4, PDE5 polypeptide, or any combination thereof.

As used herein, reference to a PDE inhibitor generally, or a specific PDE inhibitor, can include any salt, solvate, ester, or polymorph of the PDE inhibitor. A “salt” can include a pharmaceutically acceptable salt. Examples of pharmaceutically acceptable salts can include those salts prepared by reaction of a compound disclosed herein with a mineral, organic acid or inorganic base, such salts including, acetate, acrylate, adipate, alginate, aspartate, benzoate, benzenesulfonate, bisulfate, bisulfite, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate. Further, a compound disclosed herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. Other acids, such as oxalic, while not in themselves pharmaceutically acceptable, can be employed in the preparation of salts useful as intermediates in obtaining a compound and/or a pharmaceutically acceptable acid addition salt. In some embodiments, a compound disclosed herein which can comprise a free acid group reacts with a suitable base, such as the hydroxide, carbonate, bicarbonate, sulfate, of a pharmaceutically acceptable metal cation, with ammonia, or with a pharmaceutically acceptable organic primary, secondary or tertiary amine. Representative alkali or alkaline earth salts can include the lithium, sodium, potassium, calcium, magnesium, and aluminum salts and the like. Illustrative examples of bases can include sodium hydroxide, potassium hydroxide, choline hydroxide, sodium carbonate, N+(C1-4 alkyl)4, and the like. Representative organic amines useful for the formation of base addition salts can include ethylamine, diethylamine, ethylenediamine, ethanolamine, diethanolamine, piperazine and the like. It may be understood that a compound disclosed herein can also include the quaternization of any basic nitrogen-containing groups they contain. In some embodiments, water or oil-soluble or dispersible products can be obtained by such quaternization. A compound disclosed herein can be prepared as pharmaceutically acceptable salts formed when an acidic proton present in the parent compound either can be replaced by a metal ion, for example an alkali metal ion, an alkaline earth ion, or an aluminum ion; or coordinates with an organic base. In some embodiments, base addition salts can be also prepared by reacting the free acid form of a compound disclosed herein with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like. In addition, the salt forms of the disclosed compounds can be prepared using salts of the starting materials or intermediates.

“Magnitude estimation” or “ME” as used herein can refer to a measurement of the ability of a subject to determine the strength of a stimulant such as an odorant or a tastant.

“Recognition threshold” or “RT” as used herein can refer to a measurement of the ability of a subject to recognize the identity of a stimulant, such as an odorant or a tastant.

“Detection threshold” or “DT” as used herein can refer to a measurement of the ability of a subject to recognize exposure to a stimulant, such as an odorant or a tastant, as being pleasant or unpleasant.

“Hedonic” value or “H” value as used herein can refer to a measurement of a subject's reaction to a stimulant, such as an odorant or a tastant, as being pleasant or unpleasant.

The section headings used herein are for organizational purposes only and are not to be construed as limiting the subject matter described.

### Overview

Disclosed herein are methods of treatment by administering to a subject a phosphodiesterase (PDE) inhibitor, its salt, or a combination of PDE inhibitors or their salts. In some cases, a PDE inhibitor or a salt thereof can be administered with a device, such as an implantable device. In some cases, a PDE inhibitor can inhibit a PDE1, a PDE2, a PDE3, a PDE4, a PDE5 polypeptide, or a combination thereof.

PDE2 polypeptides can decrease aldosterone secretion. Such decrease may play a role in the regulation of elevated intracellular concentrations of cAMP and cGMP in platelets. Several regions of the brain can express PDE2 and rat experiments indicate that inhibition of PDE2 can enhance memory. PDE2 may play a role in regulation of fluid and cell extravasation during inflammatory conditions as PDE2 can be localized to microvessels, especially venous capillary and endothelial cells. PDE2 may also be a good pharmacological target for pathological states such as sepsis or in more localized inflammatory responses such as thrombin-induced edema formation in the lung.

The PDE3 family hydrolyzes cAMP and cGMP, but in a manner suggesting that in vivo, the hydrolysis of cAMP can be inhibited by cGMP. They can also be distinguished by their ability to be activated by several phosphorylation pathways including the PKA and PI3K/PKB pathways. PDE3A can be relatively highly expressed in platelets, as well as in cardiac myocytes and oocytes. PDE3B can be a major PDE in adipose tissue, liver, and pancreas, as well as in several cardiovascular tissues. Both PDE3A and PDE3B can be highly expressed in vascular smooth muscle cells and are likely to modulate contraction. PDE5 can be a regulator of vascular smooth muscle contraction best known as the molecular target for several well-advertised drugs used to treat erectile dysfunction and pulmonary hypertension. In the lung, inhibition of PDE5 can oppose smooth muscle vasoconstriction, and PDE5 inhibitors are in clinical trials for treatment of pulmonary hypertension.

Examples of a PDE inhibitor can include, for example, filaminast, piclamilast, rolipram, Org 20241, MCI-154, roflumilast, toborinone, posicar, lixazinone, zaprinast, sildenafil, pyrazolopyrimidinones, motapizone, pimobendan, zardaverine, siguazodan, CI-930, EMD 53998, imazodan, saterinone, loprinone hydrochloride, 3-pyridinecarbonitrile derivatives, denbufyllene, albifylline, torbafylline, doxofylline, theophylline, pentoxofylline, nanterinone, cilostazol, cilostamide, MS 857, piroximone, milrinone, aminone, tolafentrine, dipyridamole, papaverine, E4021, thienopyrimidine derivatives, triflusal, ICOS-351, tetrahydropiperazino[1,2-b]beta-carboline-1,4-dione derivatives, carboline derivatives, 2-pyrazolin-5-one derivatives, fused pyridazine derivatives, quinazoline derivatives, anthranilic acid derivatives, imidazoquinazoline derivatives, and the like. In some embodiments, a one or more PDE inhibitors, or their salts, can be formulated in or on an intranasal device. In some embodiments, the device can comprise a non-specific PDE inhibitor or its salt. In some embodiments, the device can comprise an PDE inhibitor or its salt that is selective for a PDE subtype, for example, PDE: 1, 2, 3, 4, or 5. In some embodiments, the intranasal the device does not comprise a PDE5 selective inhibitor. In some embodiments, the PDE inhibitor or its salt can be dosed at a range from about 0.001 mg to about: 1 mg, 2 mg, 3 mg, 4 mg, 5 mg, 6 mg, 7 mg, 8 mg, 9 mg, or 10 mg. In some embodiments, the dose can be a total dose released over a 24-hour period of time. For example, a dose of a PED inhibitor or a salt thereof can be about: 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, or 10 mg released in a 24-hour period. In some embodiments, a formulation can be in unit dose form. In some embodiments, a formulation can contain a second active ingredient, such as a corticosteroid, or an antihistamine, or a vasoconstrictor, or any combination thereof. In some embodiments, a formulation does not contain a second active ingredient. In some cases, a formulation can be a pharmaceutical composition, a coating, a layer of a coating, or a biodegradable device, or a bioabsorbable device. In some cases, a composition can be a pharmaceutical composition.

A PDE inhibitor can be a selective PDE inhibitor, or a non-specific PDE inhibitor. A PDE selective inhibitor can include a PDE1 selective inhibitor, PDE2 selective inhibitor, PDE3 selective inhibitor, PDE4 selective inhibitor, or PDE5 selective inhibitor. In some cases, a selective PDE inhibitor can be specific for more than one of PDE1, PDE2, PDE3, PDE4, and PDE5. A non-specific PDE can include a PDE inhibitor that inhibits at least two, three, four, or five of PDE1, PDE2, PDE3, PDE4, and PDE5.

A PDE inhibitor can inhibit cellular apoptosis by inhibiting TNF alpha, TRAIL and their metabolites. PDE inhibitors can activate the production and secretion of nitric oxide in all tissues thereby inducing vasorelaxation or vasodilation of all blood vessels including those of the peripheral blood vessels (inhibiting intermittent claudication), the distal extremities and in the penile region contributing to penile erection.

A non-specific PDE inhibitor can include theophylline, papaverine caffeine, IBMX (3-isobutyl-1-methylxanthine, aminophylline, doxophylline, cipamphylline, theobromine, pentoxifylline (oxpentifylline) and diprophylline. Theophylline sometimes referred to as a methylxanthine derivative that, when administered as described herein, can have anti-inflammatory effects on the airways that can be useful to combat the abnormal inflammation seen in asthmatics. In some cases, an anti-inflammatory effect can be achieved when theophylline is prescribed at or administered at levels that produce systemic levels of theophylline in the blood well below that which causes side effects. Patients with emphysema and chronic bronchitis can also be helped with theophylline when their symptoms are partially related to reversible airway narrowing.

A PDE1 selective inhibitor, formerly known as calcium- and calmodulin-dependent phosphodiesterases, can include eburnamenine-14-carboxylic acid ethyl ester (vinpocetine). In some cases, vinpocetine can be used to induce vasorelaxtion on cerebral smooth muscle tissue.

A PDE2 selective inhibitor can include EHNA (erythro-9-(2-hydroxy-3-nonyl) adenine), 9-(6-phenyl-2-oxohex-3-yl)-2-(3,4-dimethoxybenzyl)-purin-6-one (PDP), and BAY 60-7750.

A PDE3 selective inhibitor can include enoximone, milrinone (Primacor), amrinone, cilostamide, cilostazol (Pletal) and trequinsin. A PDE3 inhibitor when administered as described herein can produce sympathetic stimulation to increase cardiac inotropy, chronotropy and dromotropy. A PDE3 inhibitor when administered as described herein can also antagonize platelet aggregation, increase myocardial contractility, and enhance vascular and airway smooth muscle relaxation. PDE3A can be a regulator of this process. A PDE3 inhibitor when administered as described herein can effectively prevent aggregation. Cilastazol (Pletal), is approved for treatment of intermittent claudication. Its mechanism of action may involve inhibition of platelet aggregation along with inhibition of smooth muscle proliferation and vasodilation.

A PDE4 selective inhibitor can include mesembrine, rolipram, Ibudilast (i.e. a neuroprotective and bronchodilator drug that can be used in the treatment of asthma and stroke), and roflumilast (Daxas) and cilomilast (Airflo) (i.e. PDE4 selective inhibitors that can be administered for treatment of chronic obstructive pulmonary disease). A PDE4 selective inhibitor can at least partially suppress release of inflammatory mediators e.g., cytokines, or at least partially inhibit production of reactive oxygen species and immune cell infiltration. A PDE4 inhibitor can also be used to treat asthma, arthritis, and psoriasis.

A PDE5 selective inhibitor can include Sildenafil, tadalafil, vardenafil, udenafil and avanafil.

When administered in vivo, a compound, or a device described herein can be administered in combination with one or more pharmaceutically acceptable carriers or excipients and in dosages described herein. In some cases, a device can comprise a stent. A compound, or a device can be formulated as pharmaceutically acceptable a neutral (free base) or a salt form. In some embodiments, a pharmaceutically acceptable carrier can include but are not limited to: an amino acid, a peptide, a protein, a non-biological polymer, a biological polymer, a simple sugar, a carbohydrate, a gum, an inorganic salt and a metal compound which may be present singularly or in combination. In some embodiments, a pharmaceutically acceptable carrier can comprise native, derivatized, a modified form, or combinations thereof.

In some embodiments, a composition or formulation can include an excipient. Excipients can include, but are not limited to one or more of: water, a fluidizer, a lubricant, an adhesion agent, a surfactant, an acidifying agent, an alkalizing agent, an agent to adjust pH, an antimicrobial preservative, an antioxidant, an anti-static agent, a buffering agent, a chelating agent, a humectant, a or a wetting agent. Excipients can also include a coloring agent, a coating agent, a sweetening agent, a flavoring and perfuming agent or a masking agent. A composition and formulation can include a therapeutic agent with an individual excipient or with multiple excipients in any suitable combination, with or without a carrier. In some cases, an excipient can comprise glycerol.

In some cases, a pharmaceutically acceptable excipient can comprise acacia, acesulfame potassium, acetic acid, glacial, acetone, acetyl tributyl citrate, acetyl triethyl citrate, agar, albumin, alcohol, alginic acid, aliphatic polyesters, alitame, almond oil, alpha tocopherol, aluminum hydroxide adjuvant, aluminum oxide, aluminum phosphate adjuvant, aluminum stearate, ammonia solution, ammonium alginate, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylparaben, calcium alginate, calcium carbonate, calcium phosphate, dibasic anhydrous, calcium phosphate, dibasic dihydrate, calcium phosphate, tribasic, calcium stearate, calcium sulfate, canola oil, carbomer, carbon dioxide, carboxymethylcellulose calcium, carboxymethylcellulose sodium, carrageenan, castor oil, castor oil, hydrogenated, cellulose (e.g. microcrystalline, powdered, silicified microcrystalline, acetate, acetate phthalate) ceratonia, cetostearyl alcohol, cetrimide, cetyl alcohol, cetylpyridinium chloride, chitosan, chlorhexidine, chlorobutanol, chlorocresol, chlorodifluoroethane, chlorofluorocarbons, chloroxylenol, cholesterol, citric acid monohydrate, colloidal silicon dioxide, coloring agents, copovidone, corn oil, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol palmitostearate, ethylene vinyl acetate, ethylparaben, fructose, fumaric acid, gelatin, glucose, glycerin, glyceryl behenate, glyceryl monooleate, glyceryl monostearate, glyceryl palmitostearate, glycofurol, guar gum, hectorite, heptafluoropropane, hexetidine, hydrocarbons, hydrochloric acid, hydroxyethyl cellulose, hydroxyethylmethyl cellulose, hydroxypropyl cellulose, hydroxypropyl cellulose, low-substituted, hydroxypropyl starch, hypromellose, hypromellose acetate succinate, hypromellose phthalate, honey, imidurea, inulin, iron oxides, isomalt, isopropyl alcohol, isopropyl myristate, isopropyl palmitate, kaolin, lactic acid, lactitol, lactose, anhydrous, lactose, monohydrate, lactose, spray-dried, lanolin, lanolin alcohols, lanolin, hydrous, lauric acid, lecithin, leucine, linoleic acid, macrogol hydroxystearate, magnesium aluminum silicate, magnesium carbonate, magnesium oxide, magnesium silicate, magnesium stearate, magnesium trisilicate, malic acid, maltitol, maltitol solution, maltodextrin, maltol, maltose, mannitol, medium-chain triglycerides, meglumine, menthol, methylcellulose, methylparaben, mineral oil, mineral oil, light, mineral oil and lanolin alcohols, monoethanolamine, monosodium glutamate, monothioglycerol, myristic acid, neohesperidin dihydrochalcone, nitrogen, nitrous oxide, octyldodecanol, oleic acid, oleyl alcohol, olive oil, palmitic acid, paraffin, peanut oil, pectin, petrolatum, petrolatum and lanolin alcohols, phenol, phenoxyethanol, phenylethyl alcohol, phenylmercuric acetate, phenylmercuric borate, phenylmercuric nitrate, phosphoric acid, polacrilin potassium, poloxamer, polycarbophil, polydextrose, polyethylene glycol, polyethylene oxide, polymethacrylates, poly(methyl vinyl ether/maleic anhydride), polyoxyethylene alkyl ethers, polyoxyethylene castor oil derivatives, polyoxyethylene sorbitan fatty acid esters, polyoxyethylene stearates, polyvinyl acetate phthalate, polyvinyl alcohol, potassium alginate, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, 2-pyrrolidone, raffinose, saccharin, saccharin sodium, saponite, sesame oil, shellac, simethicone, sodium acetate, sodium alginate, sodium ascorbate, sodium benzoate, sodium bicarbonate, sodium borate, sodium chloride, sodium citrate dihydrate, sodium cyclamate, sodium hyaluronate, sodium hydroxide, sodium lactate, sodium lauryl sulfate, sodium metabisulfite, sodium phosphate, dibasic, sodium phosphate, monobasic, sodium propionate, sodium starch glycolate, sodium stearyl fumarate, sodium sulfite, sorbic acid, sorbitan esters (sorbitan fatty acid esters), sorbitol, soybean oil, starch, starch (e.g. pregelatinized, sterilizable maize), stearic acid, stearyl alcohol, sucralose, sucrose, sugar, compressible, sugar, confectioner's, sugar spheres, sulfobutylether b-cyclodextrin, sulfuric acid, sunflower oil, suppository bases, hard fat, talc, tartaric acid, tetrafluoroethane, thaumatin, thimerosal, thymol, titanium dioxide, tragacanth, trehalose, triacetin, tributyl citrate, triethanolamine, triethyl citrate, vanillin, vegetable oil, hydrogenated, water, wax, anionic emulsifying, wax (e.g. carnauba, cetyl esters, microcrystalline, nonionic emulsifying, white, yellow), xanthan gum, xylitol, zein, zinc acetate, zinc stearate, or any combination thereof.

In some embodiments, a device, such as a stent described herein can comprise a PDE inhibitor or a salt thereof such as theophylline or a salt thereof. In some instances, a stent can be coated with at least one layer. In some cases, a layer can comprise a PDE inhibitor or a salt thereof. In some cases, a stent can be impregnated with a PDE inhibitor or a salt thereof. In some instances, a stent can be coated and impregnated with a PDE inhibitor or a salt thereof. In some cases, a stent can be an internal device or an external device. In some cases, a device can be an internal device or an external device. In some instances, a device can be biodegradable or nonbiodegradable. In some cases, a device can be molded, printed, assembled or designed to a specific shape. In some instances, a device can be a film or comprise a core that comprises a drug, for example a PDE inhibitor. In some cases, an implant can comprise a drug surrounded by a permeable polymeric membrane that can control the release rate of the drug into the body. In some cases, an implant can be a rod, a film, a plug, a pellet, a disc, or any shape. In some cases, the stent can be an expanding stent, such that the stent expands once inserted into the desired body location. In some cases, the release kinetic of the drug can be at least partially consistent.

In some embodiments, an implant described herein can comprise a luteolin, a salt thereof, or a derivative thereof. In some cases, an implant described herein can comprise a palmitoylethanolamide, a derivative thereof, or a salt thereof. In some cases, an implant described herein can comprise a palmitoylethanolamide, a derivative thereof, or a salt thereof and a luteolin, a derivative thereof, or a salt thereof. In some cases, a palmitoylethanolamide, a derivative thereof, or a salt thereof and/or a luteolin a derivative thereof, or a salt thereof can comprise an active agent in an implant. In some cases, a palmitoylethanolamide, a derivative thereof, a salt thereof, a luteolin, a derivative thereof, or a salt thereof can be mixed with a PDE inhibitor or salt thereof. In some cases, the PDE inhibitor can comprise theophylline or a salt thereof. In some cases, a palmitoylethanolamide, a derivative thereof, or a salt thereof, and a luteolin, a derivative thereof, or a salt thereof can be mixed with a PDE inhibitor or salt thereof. In some cases, a palmitoylethanolamide, a derivative thereof, or a salt thereof and/or a luteolin a derivative thereof, or a salt thereof can be used to treat a chemosensory dysfunction. In some instances, a palmitoylethanolamide, a derivative thereof, a salt thereof, a luteolin, a derivative thereof, a salt thereof, a PDE inhibitor or a salt thereof, can each independently be present in a therapeutically effective amount to treat a chemosensory dysfunction. In some cases, a PDE inhibitor or a salt thereof can be administered intranasally and a palmitoylethanolamide, a derivative thereof, a salt thereof, a luteolin, a derivative thereof, a salt thereof, or any combination thereof can be administered orally, for example in the form of a pill, a liquid, a capsule or a tablet. In some embodiments representative dosages of a palmitoylethanolamide, a derivative thereof, a salt thereof, a luteolin, a derivative thereof, or a salt thereof can independently be from about 1.0 μg to 2000 mg per day, from about 1.0 μg to 500.0 mg per day, from about 10 μg to 100.0 mg per day, from about 10 μg to about 10 mg per day, from about 10 μg to 1.0 mg per day, from about 10 μg to 500 μg per day, from about 20 μg to about 2000 μg per day, from about 100 μg to about 10,000 μg per day, or from about 1 μg to 50 μg per day. These ranges of dosage amounts represent total dosage amounts of the active ingredient per day for a given patient. In some embodiments, a daily administered dose can be less than about: 2000 mg per day, 1000 mg per day, 500 mg per day, 100 mg per day, 10 mg per day, 1.0 mg per day, 500 μg per day, 300 μg per day, 200 μg per day, 100 μg per day or 50 μg per day. In some embodiments, a daily administered dose can be at least about: 2000 mg per day, 1000 mg per day, 500 mg per day, 100 mg per day, 10 mg per day, 1.0 mg per day, 500 μg per day, 300 μg per day, 200 μg per day, 100 μg per day or 50 μg per day. In some embodiments, on a per kilo basis, suitable dosage levels of a compound can be from about 0.001 μg/kg to about 10.0 mg/kg of body weight per day, from about 0.5 μg/kg to about 0.5 mg/kg of body weight per day, from about 1.0 μg/kg to about 100 μg/kg of body weight per day, and from about 2.0 μg/kg to about 50 μg/kg of body weight per day.

In some embodiments, a device can comprise a metal. In some cases, a device herein can comprise an alloy. In some cases, a device herein can comprise a stainless steel, a 316L stainless steel, a cobalt, a chromium, a nickel, a molybdenum, a titanium, a cobalt-chromium alloy, a nickel-titanium alloy, a platinum chromium alloy, a platinum, a tantalum alloy, or any combination thereof. In some cases, a device can comprise a cobalt alloy, a chromium alloy, a nickel alloy, a titanium alloy, or any combination thereof. In some cases, a device can comprise an iron, a magnesium or both. In some cases, a device can comprise a silicone. In some cases, a metal device can comprise a coating such as a coating that comprises an active ingredient.

In some embodiments, a device, such as an implant can comprise a polymer. In some cases, an implant can comprise a nonbiodegradable polymer such as silicone, polyvinyl alcohol (PVA), and ethylene vinyl acetate (EVA). In some cases, an implant can comprise a biodegradable polymer such as natural polymer or unnatural polymer. In some instances, an implant can comprise a nonbiodegradable polymer and a biodegradable polymer. For example, a biodegradable polymer can comprise a bovine serum albumin, a human serum albumin, a collagen, a gelatin, a polylactic acid (PLA), a polyglycolide, a poly glycolic acid (PGA), a polylactic-co-glycolic acid (PLGA), a poly(lactide-co-glycolide), a poly(L-lactide-co-ε-ca-prolactone), a polyhydroxybutyrate (PHB), a polyhydroxyvalerate (PHV), a polydioxanone, a polyglactin, a polyphosphoester, a polyanhydride, a polyphosphazene, a polycaprolactone, a polyglyconate, a poly(glycolide-co-trimethylene carbonate), a poly(sebacic acid), a poly(ester urethane), a poly(ester urethane) urea, a polyethylene glycol (PEG), or any combination thereof. In some cases, a polymer can comprise poly(DL-lactide-co-glycolide), poly(L-lactide), or both. In some cases, a polymer can comprise poly-para-dioxanone (PPD). In some cases, a polymer can comprise hydroxypropyl cellulose (HPC). In some cases, HPC can comprise HPC-H. In some cases, HPC can comprise HPC-EL, HPC-E, HPC-L, HPC-J, HPC-G, HPC-M, HPC-R, HPC-H, or any combination thereof. In some cases, a polymer can comprise a parylene. In some cases, a polymer can comprise poly(ethylene vinyl acetate). In some cases, a device can comprise one or more polymers. In some instances, a polymer can comprise a peptide. In some cases, a collagen can comprise atelocollagen. In some instances, a polymer can have porous structure such as a nanoporous structure.

In some cases, a device polymer can comprise a polydioxanone, a polycaprolactone, a polylactide-co-caprolactone, a polyvalerolactone, a polytartronic acid, a poly β-malonic acid, a polypropylene fumarate, a poly(ethylene glycol)/poly(lactic acid) copolymer, a poly(L-lactic acid-ε-caprolactone) copolymer, a poly(lactide)-poly(ethylene glycol) copolymer, or any combination thereof. In some cases, a polymer can comprise a poly[1,6-bis(carboxyphenoxy)hexane], a poly(fumaric-co-sebacic)acid, a polyanhydrides-co-imides such as a poly-trimellitylimidoglycine-co-bis(carboxyphenoxy)hexane, a polypyromellitylimidoalanine-co-1,6-bis(carboph-enoxy)-hexane, a polysebacic acid-co-1,6-bis(p-carboxyphenoxy)hexane, a poly[sebacic acid-co-1,3-bis(p-carboxyphenoxy)propane or any combination thereof.

In some embodiments, a device polymer can comprise an environmental responsive polymer. For example, an environmental responsive polymer can have controlled degradation in response to an environmental stimulus, such as a temperature change. In some cases, a polymer can be a thermo-sensitive polymer, a light-sensitive polymer, a pH sensitive polymer, an ultrasound sensitive polymer or any combination thereof. In some cases, an environmental responsive polymer can comprise poly (N-isopropylacrylamide; PNIPAAM), poly (ethylene oxide)-poly (propylene oxide)-poly (ethylene oxide) triblock copolymers (PEO-PPO-PEO), and poly (ethylene glycol)-poly (lactic acid)-poly (ethylene glycol) triblocks (PEG-PLA-PEG).

In some cases, the amount of a device polymer in an implant can comprise a weight of about: 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, or greater than 500 mg. In some instances, the amount of a device polymer in an implant can comprise a weight from about: 10 mg to about 500 mg, 10 mg to about 100 mg, 50 mg to about 250 mg, 100 mg to about 300 mg, 150 mg to about 250 mg, 200 mg to about 400 mg, or 300 mg to about 500 mg.

In some cases, a device, such as a stent, can comprise a coating. In some embodiments, a portion of the device, such as the stent, can include the coating. For example, in some embodiments, a portion of an exterior surface comprises the coating. In other embodiments, a portion of an interior surface comprises the coating. In some embodiments, an exterior surface and an interior surface comprises the coating. As a person of skill in the art will readily appreciate, a portion can include all, or substantially all, of the interior or exterior surface of the device. In some cases, a coating can comprise one or more layers. In some cases, a layer can comprise a polymer. In some cases, a layer can comprise a nonbiodegradable polymer such as silicone, polyvinyl alcohol (PVA), ethylene vinyl acetate (EVA), or a combination thereof. In some cases, a layer can comprise a biodegradable polymer such as natural polymer or unnatural polymer. In some cases, a layer can comprise a device polymer such as those described above. In some instances, a layer can comprise a nonbiodegradable polymer and a biodegradable polymer. In some cases, a layer can comprise an active pharmaceutical ingredient, for example a PDE inhibitor. In some cases, a layer can comprise a bovine serum albumin, a human serum albumin, a collagen, a gelatin, a polylactic acid (PLA), a polyglycolide (PGA), a polylactic-co-glycolic acid (PLGA), a poly(lactide-co-glycolide), a poly(L-lactide-co-ε-ca-prolactone), a polyhydroxybutyrate (PHB), a polyhydroxyvalerate (PHV), a polydioxanone, a polyglactin, a polyphosphoester, a polyanhydride, a polyphosphazene, a polycaprolactone, a polyglyconate, a poly(glycolide-co-trimethylene carbonate), a poly(sebacic acid), a poly(ester urethane), a poly(ester urethane) urea, a polyethylene glycol (PEG), or any combination thereof. In some cases, a layer can comprise poly-para-dioxanone (PPD). In some cases, a layer can comprise a parylene. In some cases, a layer can comprise poly(ethylene vinyl acetate). In some cases, a layer can comprise poly(DL-lactide-co-glycolide), poly(L-lactide), or both. In some cases, a layer can comprise one or more polymers. In some instances, a layer can comprise a peptide. In some cases, a collagen can comprise atelocollagen. In some instances, a layer can have porous structure such as a nanoporous structure. In some cases, a layer can comprise poly (N-isopropylacrylamide; PNIPAAM), poly (ethylene oxide)-poly (propylene oxide)-poly (ethylene oxide) triblock copolymers (PEO-PPO-PEO), and poly (ethylene glycol)-poly (lactic acid)-poly (ethylene glycol) triblocks (PEG-PLA-PEG).

In some embodiments, a coating can be added to a device such as an implant by dip coating, spray coating, spin coating, or any other coating method. In some cases, spin coating can comprise electrostatic spin coating.

In some cases, the amount of a coating or a plurality of coatings on an implant can comprise a weight of about: 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 12 mg, 14 mg, 16 mg, 18 mg, 20 mg, 22 mg, 24 mg, 26 mg, 28 mg, 30 mg, 32 mg, 34 mg, 36 mg, 38 mg, 40 mg, 42 mg, 44 mg, 46 mg, 48 mg, 50 mg, 55 mg, 60 mg, 65 mg, 70 mg, 75 mg, 80 mg, 85 mg, 90 mg, 95 mg, 100 mg, or greater than 100 mg. In some cases, the amount of a coating or a plurality of coatings on an implant can comprise a weight from about: 0.1 mg to about 100 mg, 1 mg to about 10 mg, 10 mg to about 25 mg, 20 mg to about 30 mg, 25 mg to about 50 mg, 30 mg to about 80 mg, or 50 mg to about 100 mg.

In some embodiments, an implant can comprise a weight of about: 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, or greater than 500 mg. In some instances, an implant can comprise a weight from about: 10 mg to about 500 mg, 10 mg to about 100 mg, 50 mg to about 250 mg, 100 mg to about 300 mg, 150 mg to about 250 mg, 200 mg to about 400 mg, or 300 mg to about 500 mg.

In some cases, a device can be a delivery system configured to release a drug for a specific time period. For example, an implant can be configured to release theophylline or a salt thereof for an extended period of time. In some cases, a device can be configured to release a drug for about: 1 day, 2 days, 3 days, 4 days, 5 days, 6 days, 7 days, 1 week, 2 weeks, 3 weeks, 4 weeks, 5 weeks, 1 month, 2 months, 2 months, 3 months, 4 months, 5 months, 6 months, 7 months, 8 months, 9 months, 10 months, 11 months, 12 months, 1 year, 2 years, 3 years, or more than 3 years. In some cases, a device can be a reservoir delivery system configured to release a drug for a specific time period. In some cases, a device can be configured to release a drug for about: 1 day to about 7 days, 2 days to about 6 days, 1 week to about 3 weeks, 2 weeks to about 5 weeks, 3 weeks to about 7 weeks, 1 month to about 12 months, 1 month to about 4 months, 2 months to about 6 months, 3 months to about 9 months, 4 months to about 12 months, 6 months to about 1 year, 6 months to about 18 months or 1 year to about 3 years.

In some cases, a device such as a stent may be deposited or placed into a specific area (e.g., a nasal cavity or a sinus cavity). In some cases, the device may degrade over time, for example a device may degrade in about: 1 day, 2 days, 3 days, 4 days, 5 days, 6 days, 7 days, 1 week, 2 weeks, 3 weeks, 4 weeks, 5 weeks, 1 month, 2 months, 2 months, 3 months, 4 months, 5 months, 6 months, 7 months, 8 months, 9 months, 10 months, 11 months, 12 months, 1 year, or more than 1 year. In some cases, the device may degrade in about: 1 day to about 7 days, 2 days to about 6 days, 1 week to about 3 weeks, 2 weeks to about 5 weeks, 3 weeks to about 7 weeks, 1 month to about 12 months, 1 month to about 4 months, 2 months to about 6 months, 3 months to about 9 months, 4 months to about 12 months, 6 months to about 1 year, or 6 months to about 18 months. In some case, a device can be placed in an area of the body and removed in about 1 day, 2 days, 3 days, 4 days, 5 days, 6 days, 7 days, 1 week, 2 weeks, 3 weeks, 4 weeks, 5 weeks, 1 month, 2 months, 2 months, 3 months, 4 months, 5 months, 6 months, 7 months, 8 months, 9 months, 10 months, 11 months, 12 months, 1 year, or more than 1 year. In some cases, a device can be placed in an area of the body and removed in about: 1 day to about 7 days, 2 days to about 6 days, 1 week to about 3 weeks, 2 weeks to about 5 weeks, 3 weeks to about 7 weeks, 1 month to about 12 months, 1 month to about 4 months, 2 months to about 6 months, 3 months to about 9 months, 4 months to about 12 months, 6 months to about 1 year, or 6 months to about 18 months.

In some cases, a device described herein can comprise an odor or a scent. For example, an odor or a scent can comprise a floral scent, a perfume, a fruity scent, a citrus scent (e.g., lemon, lime, orange), a wood scent, woody (e.g. pine or fresh cut grass), a chemical scent (e.g., ammonia, bleach), a sweet scent (e.g., chocolate, vanilla, caramel), a minty scent (e.g., and peppermint, eucalyptus and camphor), a toasted and nutty scent (e.g., popcorn, peanut butter, almonds) or a pungent scent (e.g., blue cheese, tobacco).

In some embodiments, a device can comprise a preservative. In some cases, a preservative can be methylparaben or propylparaben. In some cases, a pH of a formulation or a device can be maintained from about 4.5 to about 7.0, or from about 5.0 to about 7.0 or from about 5.5 to about 6.5. In some cases, the osmolarity of a formulation can also be adjusted to osmolarities of from about 250 to about 350 mOsm/L.

In some cases, a device can be a reservoir delivery system configured to release a drug for a specific time period. In some cases, a device can be configured to release a drug for about: 1 hour to about 24 hours, 1 hour to about 10 hours, 2 hours to about 6 hours, 5 hours to about 20 hours, 6 hours to about 22 hours, 18 hours to about 3 days, 1 day to about 7 days, 2 days to about 6 days, 1 day to about 4 days, 4 days to about 2 weeks, 1 week to about 3 weeks, 2 weeks to about 5 weeks, 3 weeks to about 7 weeks, 1 month to about 12 months, 1 month to about 4 months, 2 months to about 6 months, 3 months to about 9 months, 4 months to about 7 months, or 4 months to about 12 months.

Also disclosed herein are methods of making compositions and implants disclosed herein. In some cases, a method can comprise mixing components of a composition in a mixer or container. In some cases, a method of making can comprise contacting components of a mixture to form an implant. In some cases, an implant can be packaged in a container. In some cases, an implant can be cured in an oven. In some cases, an implant can be cured with a chemical reaction. In some cases, an implant can be cured with a light.

### Methods of Treatment

Disclosed herein are methods for treating and preventing chemosensory dysfunction in a human. In some cases, the chemosensory dysfunction can be at least in part produced from, or occurring during or after, a viral infection (e.g., a coronavirus infection, an influenza infection). In some cases, the chemosensory dysfunction can be at least in part produced from, or occurring during or after, damage to the nervous system (e.g., the sensory nervous system). In some cases, the chemosensory dysfunction can be at least in part produced from, or occurring during or after, a coronavirus infection or a mutated form thereof. In some cases, a coronavirus can comprise SARS-CoV-2 or a mutated form thereof, which can cause the disease COVID-19. In some cases, the method can comprise administering to a subject a therapeutically effective amount of a phosphodiesterase (PDE) inhibitor or a pharmaceutically acceptable salt thereof to treat chemosensory dysfunction. In some cases, administration of a PDE inhibitor can be from a device for example, an implantable stent coated with a PDE inhibitor. In some cases, administration of a PDE inhibitor can be from a device for example, an implantable stent impregnated with a PDE inhibitor. In some cases, a PDE inhibitor or pharmaceutically acceptable salt thereof can be administered in a formulation in unit dose form.

In some cases, a disease or condition described herein can be chemosensory dysfunction. In some cases, chemosensory dysfunction can comprise at least partial: loss of the sense of taste, loss of the sense of smell or both. Chemosensory dysfunction can comprise a taste or smell disorder. In some cases, a taste or smell disorder can comprise anosmia, hyposmia, ageusia, hypogeusia, dysosmia, phantosmia, dysgeusia, parosmia, or a combination thereof.

The methods of treatment can include by way of example only, oral administration, transmucosal administration, buccal administration, nasal administration such as inhalation, parental administration, intravenous, subcutaneous, intramuscular, sublingual, transdermal administration, and rectal administration. In some cases, administration can comprise administration to a nasal cavity or a sinus cavity. In some cases, administration can comprise administering a treatment as described herein to the ear, the eyes, the mouth or a combination thereof. In some instances, a sinus cavity can comprise an ethmoid sinus cavity, a maxillary sinus cavity, a frontal sinus cavity, or a sphenoid sinus cavity. In some instances, administration into a sinus cavity can comprise administration to a sinus ostium. In some cases, a nasal cavity can comprise a fossae. The nasal cavity can be described as a space that extends from the nares to the nasopharynx. The medial boundary can be described as the nasal septum, the inferior boundary can be described as the nasal floor. The lateral boundary can include the lateral nasal wall including the turbinates. The spaces between the turbinates and the lateral nasal wall can include the inferior meatus, middle meatus, superior meatus and sphenoethmoid recess and can be within the nasal cavity. The superior boundary can be defined by the skull base formed from the frontal bone, the cribiform plate of the ethmoid and the sphenoid bone. The olfactory nerves can be found in the superior aspect of the nasal cavity within the olfactory region/olfactory cleft below the cribiform plate. In some cases, a nasal cavity can comprise one or both cavities in the nose. In some cases, the nasal cavity can comprise the squamous mucosa, the olfactory mucosa, the respiratory mucosa, or a combination thereof. In some cases, the nasal cavity can comprise the superior turbinate, the middle turbinate, the inferior turbinate or a combination thereof. In some embodiments, the composition can be administered as a liquid nasal wash, an aerosol, a powder aerosol or a combination thereof. In some cases, the administering can comprise application intranasally in one nostril or both nostrils. In some cases, a device can be administered (e.g., implanted) to a naris, a nasal cavity, a sinus cavity or a combination thereof, for example a stent can be applied to the naris, a nasal cavity, or a sinus cavity. In some cases, a device can be administered to the ear, an eye, the mouth or a combination thereof. In some cases, a device can be administered transdermally, subcutaneously, topically or a combination thereof. In some embodiments, the liquid composition can be administered as a nasal wash. In some cases, administering a composition or device can be performed during, after or both during and after a viral infection.

In some embodiments, a PDE inhibitor or a salt thereof can be directly applied to the nasal or lingual epithelium as a patch or by an implantable device. These can contain at least one therapeutically active PDE inhibitor or its salt. In some cases, the formulations can further include at least one excipient that can be formulated for administration. In some cases, a device can be applied with an applicator to at least a portion of one or more: nasal cavities, sinus cavities or any combination thereof.

In some embodiments, a device described herein, such as a stent or a patch, when stored in a sealed container and placed in a room at 25° C. and a room atmosphere having about 50 percent relative humidity, can retain at least about: 80%, 90%, 95%, 96%, 97%, 98%, or 99% of the active ingredient or the salt thereof after 6 months, as measured by HPLC.

In some embodiments, the subject can be administered an olfactory assay that measures: threshold, discrimination, identification or any combination thereof. In some instances, an olfactory assay can be administered prior to, during, or after treatment. In some cases, a threshold assay can be used to determine the lowest concentration of an odorant that can be reliably detected. In some cases, a discrimination assay can be used to assess the ability of a subject to distinguish two or more different smells. In some cases, an identification assay can be used to assess the ability of a subject to identify a specific odor. In some cases, an olfactory test can be used to determine the efficacy of a treatment such as treatment with an implant comprising a PDE inhibitor. For example, an olfactory assay can be completed prior to treatment and after treatment to determine a measurable change in a subject's chemosensory dysfunction.

In some embodiments, a method of treatment can comprise olfactory training (e.g., smell training). In some cases, olfactory training, which can involve repetitive stimulation of peripheral olfactory neurons, may rely on the regenerative capacity of superior olfactory pathways. In some cases, olfactory training can comprise exposure to 1, 2, 3, 4, 5, 6 or more than 6 different odors. In some cases, olfactory training can comprise exposure to the different odors 1, 2, 3, 4 or more than 4 times per day. In some cases, olfactory training can take place for about: 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, or longer than 1 year. In some cases, olfactory training can take place for about: 7 days to about 21 days, 21 days to about 120 days, 30 days to about 90 days, 1 month to about 5 months, 2 months to about 3 months, 3 months to about 8 months, 6 months to about 1 year, 8 months to about 18 months, or 1 year to about 3 years. In some cases, organic essences of lemon, rose, eucalyptus and cloves can be used as the different odors for olfactory testing

In some embodiments, a subject can experience a change (e.g., a decrease) in: a detection threshold (DT) score, a recognition threshold (RT) score, or both. In some embodiments, a subject can experience a change (e.g., an increase) in a magnitude estimation (ME) score. In some cases, a subject can experience a change in a hedonic (H) score. In some cases, the changes in RT score, ME score, DT score, or H score can be measured with a forced-choice, three-stimuli, stepwise-staircase technique using one or more odorants after the administration of a PDE inhibitor to a subject. In some cases, the one or more odorants comprise pyridine, nitrobenzene, thiophene, amyl acetate, or a combination thereof. In certain embodiments, a DT score, RT score, ME score, or H score can be used to assess whether a subject is improving or responding to a treatment. For example, in some cases, a DT score and/or RT score can be measured before and after administration of a PDE inhibitor. The DT score and/or RT score can be measured using a staircase technique with an odorant. If the measured score decreases after the administration of the PDE inhibitor, the health status of the subject can be identified as improving.

In some embodiments, the subject can experience a change (e.g., a decrease) in: a taste detection threshold (DT) score, a recognition threshold (RT) score, or both. In some embodiments, a subject can experience a change (e.g., an increase) in a magnitude estimation (ME) score. In some cases, a subject can experience a change in a hedonic (H) score. In some cases, the changes in RT score, ME score, DT score, or H score can be measured with a forced-choice, three-stimuli, stepwise-staircase technique using one or more tastants testing compounds after administration of a PDE inhibitor to the subject. In some cases, the one or more tastants comprise sodium chloride (NaCl), sucrose, hydrogen chloride (HCl), urea, or a combination thereof.

In some embodiments, a subject can experience a clinically detectable improvement in taste or smell function within about: 1 week to about 6 weeks, 1 week to about 4 weeks, 2 weeks to about 5 weeks, or about 3 weeks to about 4 weeks of starting treatment. In some embodiments, a subject can experience a clinically detectable improvement in taste or smell function within about: 1 month to about 6 months, 1 month to about 4 months, 2 months to about 5 months, or about 3 months to about 4 months of starting treatment.

In some cases, administering a PDE inhibitor as described herein can be used to prevent or treat diseases or conditions associated with or caused by a viral infection, a bacterial infection, a fungal infection, a parasitic infection or any combination thereof. In some cases, administering a PDE inhibitor as described herein can be used to prevent or treat diseases or conditions associated with the nervous system, such as the sensory nervous system, the central nervous system, or the peripheral nervous system. In some cases, a viral infection can comprise coronavirus. In some cases, a viral infection can comprise an influenza virus. Such diseases and conditions can include, for example, anosmia, taste loss, smell loss, hyposmia, ageusia, dysosmia, parosmia, phantosmia, chemosensory dysfunction, cough, fever, fever, malaise, difficult breathing, runny nose, sore throat, nasal congestion or any combination thereof. In some cases, an influenza infection can be caused by Influenza A, Influenza B (e.g., B (Victoria), B (Yamagata)). In some cases, Influenza A can comprise Influenza H1N1, H3N2, a mutation of any of these, or any combination thereof. In some cases, an influenza virus can comprise one or more mutations. In some cases, a coronavirus can cause COVID-19. In one example, an implant described herein can be used to treat chemosensory dysfunction resulting from SARS-CoV-2 or a mutated form thereof. In some cases, a coronavirus infection can be caused by alpha coronavirus, beta coronavirus, gamma coronavirus, delta coronavirus, 229E coronavirus, NL63 coronavirus, OC43 coronavirus, HKU1 coronavirus, MERS-CoV, SARS-CoV, SARS-CoV-2, a mutated form thereof, or any combination of these. In some cases, a virus, such as a coronavirus can have a mutation. In some cases, a coronavirus or an influenza virus can comprise a genome with more than about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity or sequence similarity as compared to a reference genome sequence. In some cases, a coronavirus or an influenza virus can comprise a genome with less than about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity or sequence similarity as compared to a reference genome sequence. In some cases, a virus, such as a coronavirus or an influenza virus can have a mutation. For example, a coronavirus or an influenza virus can comprise about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotide mutations as compared to a reference sequence. In some cases, a reference sequence can be a reference sequence from the National Center for Biotechnology Information.

In some cases, methods or compositions of the disclosure as described herein can be used to treat a subject inflicted with a chemosensory disorder. A chemosensory disorder can include a loss of smell (anosmia), or a reduced ability to smell (hyposmia). A chemosensory disorder can include a loss of taste (ageusia), or a reduced ability to taste (hypogeusia), for example, a reduced ability to taste a sweet, sour, bitter, or salty substance. In some instances, a chemosensory disorder involves odors or tastes or flavors to be misread or distorted. For example, a chemosensory disorder can cause a person to detect an unpleasant odor or taste from something that is normally pleasant to taste or smell. A chemosensory disorder (e.g., can accompany, or be associated with, a disease or condition, such as, obesity, diabetes, hypertension, malnutrition, or a degenerative disease of the nervous system, such as, Parkinson's disease, Alzheimer's disease, or multiple sclerosis.

Disclosed herein are methods of treating a condition (e.g., taste and smell loss) by administering a PDE inhibitor or salt thereof as described herein. In some cases, administering can comprise administering a PDE inhibitor or salt thereof in unit dose form. In some cases, administering a PDE inhibitor or salt thereof can comprise administering a device such as a stent that delivers the PDE inhibitor for an extended period of time. In some embodiments representative daily intranasal, lingual, pulmonary, topical or mucosal dosages are from about 1.0 μg to 2000 mg per day, from about 1.0 μg to 500.0 mg per day, from about 10 μg to 100.0 mg per day, from about 10 μg to about 10 mg per day, from about 10 μg to 1.0 mg per day, from about 10 μg to 500 μg per day or from about 1 μg to 50 μg per day of the active ingredient (e.g., a PDE inhibitor). These ranges of dosage amounts represent total dosage amounts of the active ingredient per day for a given patient. In some embodiments, a daily administered dose can be less than about: 2000 mg per day, 1000 mg per day, 500 mg per day, 100 mg per day, 10 mg per day, 1.0 mg per day, 500 μg per day, 300 μg per day, 200 μg per day, 100 μg per day or 50 μg per day of the active ingredient. In some embodiments, a daily administered dose can be at least about: 2000 mg per day, 1000 mg per day, 500 mg per day, 100 mg per day, 10 mg per day, 1.0 mg per day, 500 μg per day, 300 μg per day, 200 μg per day, 100 μg per day or 50 μg per day of the active ingredient. In some embodiments, on a per kilo basis, suitable dosage levels of a compound can be from about 0.001 μg/kg to about 10.0 mg/kg of body weight per day, from about 0.5 μg/kg to about 0.5 mg/kg of body weight per day, from about 1.0 μg/kg to about 100 μg/kg of body weight per day, and from about 2.0 μg/kg to about 50 μg/kg of body weight per day. In some embodiments, a suitable dosage level of an active ingredient on a per kilo basis can be less than about: 10.0 mg/kg of body weight per day, 1 mg/kg of body weight per day, 500 μg/kg of body weight per day, 100 μg/kg of body weight per day, 10 μg/kg of body weight per day, or 1.0 μg/kg of body weight per day. In some embodiments, a suitable dosage level of the active ingredient on a per kilo basis can be at least about: 10.0 mg/kg of body weight per day, 1 mg/kg of body weight per day, 500 μg/kg of body weight per day, 100 μg/kg of body weight per day, 10 μg/kg of body weight per day, or 1.0 μg/kg of body weight per day.

In some cases, the amount administered can be the same amount administered to treat a particular disease or can be an amount lower than the amount administered to treat that particular disease. The dosage may be administered once per day or several or multiple times per day. In another example, a PDE inhibitor or salt thereof can be administered 2, 3, 4, 5, 6, 7, 8, 9, 10 or more times per day. In some cases, a composition can be administered once, twice or thrice in a 24-hour period. In some cases, a device can be configured to release a PDE inhibitor or salt thereof for a period of time. In some cases, a device can be configured for extended release of a PDE inhibitor or salt thereof. In some cases, a device can be configured to release an active agent with substantially zero order kinetics. In some cases, a device can be configured to release an active agent with substantially first order kinetics. In some cases, a device can be implanted once in a lifetime, or as needed. For example, a device can be implanted every: 1 day, 2 days, 3 days, 4 days, 5 days, 6 days, 7 days, 1 week, 2 weeks, 3 weeks, 4 weeks, 5 weeks, 1 month, 2 months, 2 months, 3 months, 4 months, 5 months, 6 months, 7 months, 8 months, 9 months, 10 months, 11 months, 12 months, 1 year, 2 years, 3 years, or more than 3 years. In some cases, a PDE inhibitor or salt thereof, or a formulation, or a device comprising a PDE inhibitor or salt thereof can be administered about every: 1 day, 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, or for life. The amount of the drug administered to practice methods of the present disclosure will of course, be dependent on the subject being treated, the severity of the affliction, the manner of administration and the judgment of the prescribing physician. The dose used to practice the disclosure can produce the desired therapeutic or prophylactic effects, without producing serious side effects.

In some embodiments, administration of an effective amount of a PDE inhibitor by intranasal, lingual, pulmonary, topical, device mediated, or mucosal administration does not produce a detectable blood level of the PDE inhibitor. In some embodiments, administration of an effective amount of a PDE inhibitor by intranasal, lingual, pulmonary, topical, device mediated, or mucosal administration produces blood concentration of the PDE inhibitor that are less than about: 5 mg/dl, 2 mg/dl, 1 mg/dl, 500 μg/dl, 250 μg/dl, 100 μg/dl, 50 μg/dl, 25 μg/dl, 10 μg/dl, 5 μg/dl, or 1 μg/dl. In some embodiments, administration of an effective amount of a PDE inhibitor by intranasal, lingual, pulmonary, topical, device mediated, or mucosal administration produces blood concentration of the PDE inhibitor that are more than about: 2 mg/dl, 1 mg/dl, 500 μg/dl, 250 μg/dl, 100 μg/dl, 50 μg/dl, 25 μg/dl, 10 μg/dl, 5 μg/dl, or 1 μg/dl.

In some embodiments, administration of an effective amount of a PDE inhibitor or salt thereof can increase the salivary and/or nasal mucus cAMP or cGMP levels in the human by at least about: 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or about 50% relative to these levels in the human before the administering of the therapeutically effective amount of PDE inhibitor or pharmaceutically acceptable salt thereof. In some cases, the increase of the salivary and/or nasal mucus cAMP or cGMP levels are observed after about: 1 to about 10 days, 30 to about 90 days, 15 to about 45 days, or 30 days of continuous treatment with the therapeutically effective amount of PDE inhibitor or pharmaceutically acceptable salt thereof.

In some embodiments, administration of an effective amount of a PDE inhibitor or a salt thereof by a device can increase taste or smell acuity. In some embodiments, an increase in taste or smell acuity can be at least about: 5%, 10%, 20%, 30%, 40%, 50%, 75%, or 100% compared to the untreated state. In some embodiments, taste or smell acuity can be increased to at least about: 5%, 10%, 15%, 20%, 25%, 30%, 35%, 40%, 45%, 50%, 55%, 60%, 6%, 70%, 75%, 80%, 85%, 90%, 95% or 100% of the acuity of normal individuals. In some cases, an increase in taste or smell acuity can be measured after about: 10 to about 20 days, 15 to about 30 days, 25 days to about 50 days, 1 month to about 6 months, 4 months to 12 months, 6 months to 18 months, or 6 months to about 3 years. In some cases, an increase in taste or smell acuity can be measured after about 30 days. In some embodiments, taste or smell acuity can be measured objectively. In some embodiments, taste or smell acuity can be measured subjectively. In some cases, smell acuity can be measured by detection threshold, recognition threshold, hedonics, magnitude estimation or any combination thereof.

In some cases, a composition described herein can be administered with one or more additional therapeutics. For example, a PDE inhibitor or salt thereof can be administered with a second therapy. In some cases, a second therapy can be administered concurrently or consecutively. In some cases, an additional therapeutic can comprise an antihistamine, diphenhydramine, chlorpheniramine, cetirizine, desloratadine, fexofenadine, levocetirizine, loratadine, azelastine, olopatadine, ketotifen, olopatadine, pheniramine, a decongestant, pseudoephedrine, oxymetazoline, tetrahydrozoline, a corticosteroid, budesonide, fluticasone furoate, fluticasone propionate, mometasone, triamcinolone, beclomethasone, ciclesonide, budesonide, fluticasone, mometasone, fluorometholone, loteprednol, prednisone, betamethasone, desonide, hydrocortisone, mometasone, triamcinolone, cromolyn, lodoxamide, nedocromil, an immunotherapy, a salt of any of these, or any combination thereof. In some cases, a second therapy can comprise remdesivir, a salt thereof, chloroquine, a salt thereof, lopinavir, a salt thereof, ritonavir, a salt thereof, favilavir, a salt thereof, interferon beta, a salt thereof, an antiviral, oxygen or any combination thereof. In some cases, an additional therapeutic can comprise nitric oxide, a steroid, a non-steroidal anti-inflammatory drug (NSAID), or any combination thereof. In some cases, an additional therapy can be comprised in a device that comprises a PDE inhibitor.

In some cases, a subject can be diagnosed (e.g., diagnosed with chemosensory dysfunction) prior to treatment with a PDE inhibitor or salt thereof. In some cases, a method of treatment can comprise diagnosing chemosensory dysfunction in a subject. In some cases, the diagnosing can comprise an in vitro assay. In some cases, chemosensory dysfunction can be diagnosed by detecting sonic hedgehog at or below a threshold level in a biological sample from the human. In some cases, chemosensory dysfunction can be diagnosed by cyclic AMP (cAMP), cyclic GMP (cGMP), or both at or below a threshold level in a biological sample from the human. In some cases, a sample can be a nasal sample or a saliva sample. In some cases, chemosensory dysfunction can be diagnosed by detecting a cyclic nucleotide level at or below a threshold level in the biological sample from the human. In some cases, chemosensory dysfunction can be diagnosed by determining a detection threshold, by determining a recognition threshold, and by magnitude estimation for at least one of: pyridine, nitrobenzene, thiophene, and amyl acetate. In some cases, diagnosis can comprise detecting a RT score, a ME score, a DT score, a H score or a combination thereof and comparing to a reference population (e.g., a population without chemosensory dysfunction).

The Hedgehog signaling pathway is sometimes referred to as a key regulator of animal development, particularly during late stages of embryogenesis and metamorphosis. Mammals may have three Members of the hedgehog signaling pathway, Sonic Hedgehog (SHH), Desert Hedgehog (DHH), and Indian hedgehog (IHH). The pathway may be implicated in the development of some cancers. Members of the hedgehog signaling pathway can be used in diagnosing and treating loss and/or distortion of taste or smell, e.g., hyposmia, dysosmia, anosmia, phantosmia, hypogeusia, dysgeusia, phantogeusia, and/or ageusia.

The term “one or more members of the hedgehog signaling pathway” as used herein and its grammatical equivalents can include known or unknown members of the hedgehog signaling pathway. For example, known members of the hedgehog signaling pathway can include the currently known members of the hedgehog signaling pathway, Sonic Hedgehog (SHH), Desert Hedgehog (DHH), and Indian hedgehog (IHH). Unknown members of the hedgehog signaling pathway can be found by comparing the homology of nucleic acid and proteins sequences. Although, the disclosure is directed towards any member of the hedgehog signaling pathway, specific hedgehog members can be of significant influence. Therefore, it is contemplated that the disclosure can focus on SHH, DHH, IHH, or any combination thereof. For example, the embodiments disclosed herein can be focused on SHH.

The one or more members of the hedgehog signaling pathway can be selected from a group consisting of: Sonic Hedgehog (SHH), Desert Hedgehog (DHH), and/or Indian hedgehog (IHH). The one or more members of the hedgehog signaling pathway can be SHH, DHH, IHH, or any combination thereof. Although a mammalian (e.g., human) hedgehog can be measured, it is also contemplated that a non-mammalian hedgehog can be measured. In some cases, chemosensory dysfunction in a subject can be determined by detecting a level of: Sonic Hedgehog (SHH) that ranges from about greater than 0 pg/mL to about 8,500 pg/mL; a level of Indian hedgehog (IHH) that ranges from about greater than 0 pg/mL about to 1.0 pg/mL; or a level of Desert Hedgehog (DHH) that ranges from about greater than 0 pg/mL to about 5.0 pg/mL, or a combination thereof.

The levels of members of the hedgehog signaling pathway in patients exhibiting loss and/or distortion of taste or smell (e.g., hyposmia, dysosmia, anosmia, phantosmia, hypogeusia, dysgeusia, phantogeusia, and/or ageusia) can be lower than normal controls. For example, in patients suffering from loss and/or distortion of taste or smell (e.g., chemosensory dysfunction), the level of SHH, in some cases, will be or about: 0 pg/mL, greater than 0 pg/mL to less than less than 1 pg/mL, 1 pg/mL to 25 pg/mL, 15 pg/mL to 30 pg/mL, 20 pg/mL to 40 pg/mL; 35 pg/mL to 50 pg/mL; 45 pg/mL to 100 pg/mL; 75 pg/mL to 150 pg/mL, 125 pg/mL to 1000 pg/mL, 900 pg/mL to 2500 pg/mL, 2000 pg/mL to 5000 pg/mL, 4000 pg/mL to 7500 pg/mL, 6000 pg/mL to 10,000 pg/mL; (b) the level of IHH can be or about: 0 pg/mL, greater than 0 pg/mL to 0.1 pg/mL, 0.05 pg/mL to 0.15 pg/mL, 0.125 pg/mL to 0.2 pg/mL, 0.15 pg/mL to 0.30 pg/mL, 0.25 pg/mL to 0.5 pg/mL, 0.4 pg/mL to 0.7 pg/mL, 0.6 pg/mL to 0.75 pg/mL, 0.725 pg/mL to 0.9 pg/mL, 0.8 pg/mL to 1.0 pg/mL, less than 1.0 pg/mL, less than 0.05 ng/mL, less than 0.15 ng/mL, less than 0.2 ng/mL, less than 0.3 ng/mL, less than 0.5 ng/mL, less than 0.7 ng/mL, less than 0.75 ng/mL, less than 0.9 ng/mL, less than 1.0 ng/mL, less than 1.1 ng/mL, less than 1.5 ng/mL, less than 1.75 ng/mL, less than 2.0 ng/mL, less than 2.25 ng/mL, less than 5.0 ng/mL, less than 6.0 ng/mL, less than 7.0 ng/mL, less than 10.0 ng/mL, or less than 100.0 ng/mL; (c) the level of DHH can be or about: 0 pg/mL, greater than 0 pg/mL to 0.1 pg/mL, 0.05 pg/mL to 0.15 pg/mL, 0.125 pg/mL to 0.2 pg/mL, 0.15 pg/mL to 0.30 pg/mL, 0.25 pg/mL to 0.5 pg/mL, 0.4 pg/mL to 0.7 pg/mL, 0.6 pg/mL to 0.75 pg/mL, 0.725 pg/mL to 0.9 pg/mL, 0.8 pg/mL to 1.0 pg/mL, 0.9 pg/mL to 1.1 pg/mL, 1.0 pg/mL to 1.3 pg/mL, 1.2 pg/mL to 1.5 pg/mL, 1.4 pg/mL to 2.0 pg/mL, 1.9 pg/mL to 2.5 pg/mL, 2.4 pg/mL to 3.0 pg/mL, 2.9 pg/mL to 3.5 pg/mL, 3.4 pg/mL to 3.8 pg/mL, 3.7 pg/mL to 3.9 pg/mL, 3.85 pg/mL to 5.0 pg/mL, less than 5.0 pg/mL, less than 0.05 ng/mL, less than 0.15 ng/mL, less than 0.2 ng/mL, less than 0.3 ng/mL, less than 0.5 ng/mL, less than 0.7 ng/mL, less than 0.75 ng/mL, less than 0.9 ng/mL, less than 1.0 ng/mL, less than 1.1 ng/mL, less than 1.5 ng/mL, less than 1.75 ng/mL, less than 2.0 ng/mL, less than 2.25 ng/mL, less than 5.0 ng/mL, less than 6.0 ng/mL, less than 7.0 ng/mL, less than 10.0 ng/mL, or less than 100.0 ng/mL. However, there can be some inter-patient variability because the levels of the different members of the hedgehog signaling pathway vary based on the person.

In some embodiments, administration of an effective amount of a PDE inhibitor (e.g., by an implant) can increase the salivary and/or nasal mucus SHH, DHH, and/or IHH levels in the human by at least about: 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or about 50% relative to these levels in the human before the administering of the therapeutically effective amount of PDE inhibitor. In some cases, the increase of the salivary and/or nasal mucus SHH, DHH, and/or IHH levels are observed after about: 1 to about 10 days, 30 to about 90 days, 15 to about 45 days, or 30 days of continuous treatment with the therapeutically effective amount of a PDE inhibitor.

In some embodiments, administration of an effective amount of a PDE inhibitor (e.g., by an implant) can increase or decrease a biological compound, such as a protein or a metabolite. In some embodiments, administration of an effective amount of a PDE inhibitor can increase the salivary and/or nasal mucus cAMP or cGMP levels in the human by at least about: 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or about 50% relative to these levels in the human before the administering of the therapeutically effective amount of the PDE inhibitor. In some cases, the increase of the salivary and/or nasal mucus cAMP or cGMP levels are observed after about: 1 to about 10 days, 30 to about 90 days, 15 to about 45 days, or 30 days of continuous treatment with the therapeutically effective amount of a PDE inhibitor.

In some embodiments, administration of an effective amount of a PDE inhibitor (e.g., by an implant) can decrease the salivary and/or nasal mucus IL-10 levels in the human by at least about: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or about 50% relative to these levels in the human before the administering of the therapeutically effective amount of the PDE inhibitor. In some cases, the decrease of the salivary and/or nasal mucus IL-10 levels are observed after about: 1 to about 10 days, 30 to about 90 days, 15 to about 45 days, or 30 days of continuous treatment with a therapeutically effective amount of a PDE inhibitor. IL-10 levels can be measured with an enzyme-linked immunoassay (ELISA), Western blot, or other protein measurement assay.

To assess if a subject has loss and/or distortion of taste or smell, e.g., hyposmia, dysosmia, anosmia, phantosmia, hypogeusia, dysgeusia, phantogeusia, and/or ageusia, based on levels of one or more members of the hedgehog signaling pathway, a threshold comparison, e.g., a basal level can be used. Thus, the threshold level can be an average level for one or more members of the hedgehog signaling pathway as measured in a control population comprising subjects with normal olfactory and/or gustatory function. The level of one or more members of the hedgehog signaling pathway can be at least one order of magnitude lower than said threshold level. For example, 2, 3, 4, 5, 10, 15, 16, 17, 18, 19, 20, 21, 22, 23, 24, 25, 26, 26, 27, 28, 29, 30, 31, 32, 33, 34, 35, 36, 37, 38, 39, 40, 41, 42, 43, 44, 45, 50, 60, 70 80, 90, 100, 150, 200, 250, 300, 400, 500, 600, 700, 800, 900, or 1000 or more orders of magnitude lower than said threshold level.

Also described herein are kits comprising a device, an applicator, or both described herein. In some cases, a kit can comprise a container that comprises a device, an applicator or both. In some instances, a kit can comprise instructions for use. In some instances, a container can be a sterile container. In some cases, a container can be a plastic, a glass, or a metal container.

### Numbered Embodiments

A number of compositions, methods, and kits are disclosed herein. Specific exemplary embodiments of these of compositions, methods, and kits are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. A method of treating or preventing a chemosensory dysfunction in a subject the method comprising: delivering a stent to a nasal or sinus cavity to treat or prevent the chemosensory dysfunction in the subject, wherein the nasal or sinus cavity comprises an inferior meatus, a middle meatus, a superior meatus, a sphenoethmoid recess, an olfactory cleft, an ethmoid sinus cavity, a maxillary sinus cavity, a frontal sinus cavity, or a sphenoid sinus cavity, wherein the stent: (a) is at least partially impregnated with a phosphodiesterase (PDE) inhibitor or a salt thereof; (b) comprises at least one coating layer on at least a portion of the stent, the coating layer comprising the PDE inhibitor or the salt thereof; or (c) both (a) and (b), wherein the at least the portion of the stent is configured to release the PDE inhibitor or the salt thereof, and wherein the PDE inhibitor or the salt thereof comprises theophylline or a salt thereof, cilostazol or a salt thereof, or roflumilast or a salt thereof, or any combination thereof.

Embodiment 2. The method of embodiment 1, wherein the stent is placed within the nasal cavity, within a sinus cavity or at a sinus ostium.

Embodiment 3. The method of embodiment 1, wherein the stent comprises the PDE inhibitor or the salt thereof which comprises theophylline or a salt thereof.

Embodiment 4. The method of embodiment 1, wherein the stent comprises the PDE inhibitor or the salt thereof which comprises cilostazol or a salt thereof.

Embodiment 5. The method of any one of embodiments 1-4, wherein the stent comprises the coating layer, and wherein the coating layer comprises a release rate modifier.

Embodiment 6 The method of embodiment 5, wherein the release rate modifier comprises a polyethylene glycol (PEG).

Embodiment 7. The method of embodiment 5, wherein the release rate modifier comprises a bovine serum albumin, a human serum albumin, a collagen, a gelatin, a polylactic acid (PLA), a polyglycolide (PGA), a polylactic-co-glycolic acid (PLGA), a poly(lactide-co-glycolide), a poly(L-lactide-co-ε-ca-prolactone), a polyhydroxybutyrate (PHB), a polyhydroxyvalerate (PHV), a polydioxanone, a polyglactin, a poly caprolactone, a polyglyconate, a poly(glycolide-co-trimethylene carbonate), a poly(sebacic acid), a poly(ester urethane), a poly(ester urethane) urea, PEG, or any combination thereof.

Embodiment 8. The method of any one of embodiments 1-7, wherein the stent comprises the at least one coating layer, and wherein the stent further comprises a second coating layer.

Embodiment 9. The method of embodiment 8, further comprising a plurality of three or more coating layers.

Embodiment 10. The method of any one of embodiments 1-9, wherein the stent is a bioabsorbable stent.

Embodiment 11. The method of any one of embodiments 1-9, wherein the stent is a non-bioabsorbable stent.

Embodiment 12. The method of any one of embodiments 1-11, wherein the stent comprises a PHB, a PHV, a polycaprolactone, or any combination thereof.

Embodiment 13. The method of any one of embodiments 1-12, wherein the stent is configured to release the PDE inhibitor or the salt thereof over a period of time from about 5 days to about 180 days.

Embodiment 14. The method of any one of embodiments 1-13, wherein the stent is configured to release about 500 μg of the PDE inhibitor or salt thereof per day.

Embodiment 15. The method of any one of embodiments 1-14, wherein the chemosensory dysfunction is ageusia, hypogeusia, dysgeusia, parosmia, phantosmia, anosmia, hyposmia, dysosmia, or any combination thereof.

Embodiment 16. The method of any one of embodiments 1-14, wherein the chemosensory dysfunction is anosmia, hyposmia, dysosmia, parosmia, or a combination thereof.

Embodiment 17. The method of any one of embodiments 1-14, wherein the chemosensory dysfunction is smell loss, taste loss, or a combination thereof.

Embodiment 18. The method of any one of embodiments 1-17, wherein the subject before treatment has a decreased level of a cyclic nucleotide, a decreased level of a sonic hedgehog, or both, in a nasal mucus sample from the subject in comparison to a reference level associated with normal chemosensory function.

Embodiment 19. The method of any one of embodiments 1-18, further comprising administering a second therapeutic.

Embodiment 20. The method of embodiment 19, wherein the second therapeutic is administered concurrently or consecutively.

Embodiment 21. The method of any one of embodiments 1-20, further comprising diagnosing the subject with chemosensory dysfunction.

Embodiment 22. The method of any one of embodiments 1-20, wherein the subject was previously diagnosed with chemosensory dysfunction.

Embodiment 23. An implantable stent comprising: a phosphodiesterase (PDE) inhibitor or a salt thereof, wherein the PDE inhibitor or the salt thereof is: (a) impregnated in at least a portion of the stent; (b) comprised in a coating layer on at least a portion of the stent; or (c) both (a) and (b), wherein the stent is configured to release the PDE inhibitor or the salt thereof in a nasal cavity of a subject and wherein the PDE inhibitor or the salt thereof comprises theophylline, a salt thereof, cilostazol, a salt thereof, or any combination thereof.

Embodiment 24. A method of treating or preventing a chemosensory dysfunction in a subject the method comprising: delivering and depositing an implantable device to a nose to treat or prevent the chemosensory dysfunction in the subject, wherein the implantable device: (a) is at least partially impregnated with a phosphodiesterase (PDE) inhibitor or a salt thereof; (b) comprises at least one coating layer on at least a portion of the implantable device, wherein the coating layer comprises the PDE inhibitor or the salt thereof; or (c) both (a) and (b), wherein the implantable device is configured to release the PDE inhibitor or the salt thereof, and wherein the PDE inhibitor or the salt thereof comprises theophylline, a salt thereof, cilostazol, a salt thereof or any combination thereof.

Embodiment 25. The method of embodiment 24, wherein the nose comprises a sinus cavity or a nasal cavity.

Embodiment 26 The method of embodiment 25, wherein the nose comprises a sinus cavity and wherein the sinus cavity comprises an ethmoid sinus cavity, a maxillary sinus cavity, a frontal sinus cavity, or a sphenoid sinus cavity.

Embodiment 27. The method of embodiment 25, wherein the nose comprises a nasal cavity and the nasal cavity comprises a fossae.

Embodiment 28. A method of making an implantable stent comprising: (a) impregnating at least a portion of a stent with a PDE inhibitor or a salt thereof; (b) coating at least a portion of a stent with a layer comprising the PDE inhibitor or the salt thereof; or (c) both (a) and (b), wherein the stent is configured to release the PDE inhibitor or the salt thereof in a nasal cavity of a subject, and wherein the PDE inhibitor or the salt thereof comprises theophylline, a salt thereof, cilostazol, a salt thereof, or any combination thereof.

Embodiment 29. A kit comprising the stent of embodiment 23.

### EXAMPLES

### Example 1

A patient diagnosed or previously diagnosed with influenza and having at least partial smell or taste loss associated with influenza is treated with an intranasally administered stent coated with theophylline. The stent is coated with a poly(lactide-co-glycolide) (PLGA) and poly(ethylene vinyl acetate) (PEVA) mixture which comprises theophylline. The coating is configured to release theophylline in an amount of about 15 μg to 150 μg per day for over 30 days.

Administration of the stent produces an increase in the patient's sonic hedgehog levels or cyclic nucleotide levels within about 30 days, 60 days, 90 days, or 180 days. Further, this increase produces a significant increase in the patient's ability to taste and smell, thus at least partially ameliorating the smell and taste loss.

### Example 2

A patient diagnosed or previously diagnosed with chemosensory dysfunction is treated with an intranasally administered biodegradable stent comprising theophylline. The stent is a molded product developed to fit at least partially inside a sinus cavity. The stent comprises a poly-para-dioxanone (PPD) mixture and theophylline. The biodegradable stent is configured degrade and release theophylline at a dosage of about 0.25 μg/kg to 2.6 μg/kg per day for over 60 days.

Administration of the stent produces an increase in the patient's sonic hedgehog levels or cyclic nucleotide levels within about 60 days. Further, this increase produces a significant increase in the patient's ability to taste and smell, thus at least partially ameliorating the chemosensory dysfunction.

### Example 3

A patient diagnosed or previously diagnosed with dysgeusia is treated with an intranasally administered stent comprising theophylline. The stent is administered to a sinus cavity of the patient. The stent comprises 2 layers: a poly(DL-lactide-co-glycolide) and poly(L-lactide) layer which comprises theophylline and a PLGA layer which comprises theophylline. The layers are configured to release theophylline at a dosage of about 15 μg to about 300 μg per day for about 90 days.

Administration of the stent produces an increase in the patient's recognition threshold and detection threshold within about 60 days. Further, this increase produces a significant increase in the patient's ability to taste, thus at least partially ameliorating the dysgeusia.

### Example 4

A patient diagnosed or previously diagnosed with COVID-19 and having at least partial chemosensory dysfunction associated with COVID-19 is treated with an intranasally administered stent coated with theophylline. The stent is coated with a poly(lactide-co-glycolide) (PLGA) and poly(ethylene vinyl acetate) (PEVA) mixture which comprises theophylline. The coating is configured to release theophylline in an amount of about 15 μg to 150 μg per day for over 30 days.

Administration of the stent produces an increase in the patient's sonic hedgehog levels or cyclic nucleotide levels within about 30 days, 60 days, 90 days, or 180 days. Further, this increase produces a significant increase in the patient's ability to taste and smell, thus at least partially ameliorating the chemosensory dysfunction.

### Example 5

The Patient initially reported their sensory dysfunction as either loss of taste (i.e., flavor) and/or smell function. This subjective response was documented by objective psychophysical measurements of olfactory function administered to each patient by use of a forced-choice, three-stimuli, stepwise-staircase technique in a fixed, controlled design (1, 2). Efficacy of this technique and results of testing were previously documented in a double-blind clinical trial (2). Four odors were used; they were pyridine (dead-fish odor), nitrobenzene (bitter-almond odor), thiophene (petroleum-like odor) and amyl acetate (banana-oil odor). Detection thresholds (DT), and recognition thresholds (RT) values for each odor were determined as previously described (1, 2). Thresholds were converted into bottle units (BU) as previously described (2) and results reported as M of correct responses for each odor in each treatment group. References: (1) Henkin, R. I. Evaluation and treatment of human olfactory dysfunction, in Otolaryngology (English, G. M. Ed.), Lip pincott, Philadelphia, 1993, Vol. 2, pp. 1-86. (2) Henkin, R. I., Schecter, P. J., Friedewald, W. T., DeMets, D. L., Raff, M. S. A double blind study of the effects of zinc sulfate on taste and smell dysfunction. Amer. J. Med. Sci. 1976; 272: 285-299.

Theophylline was administered to a patient who had loss of taste and smell from COVID-19. A formulation containing 80 μg of theophylline was administered via two actuations from nasal spray device once per day. Prior to the initial administration of theophylline, a baseline smell test was performed. A follow-up smell test was performed 3 months after the initial administration. No side effects were reported. The results are shown in Table 1. The patient's detection threshold (DT) and recognition threshold (RT) for pyridine (Pyr), nitrobenzene (NO2B), thiophene (Thio) and amyl acetate (AA) was determined before treatment (pre) and after treatment (3M). In all four odorant tests, the patient's detection threshold and recognition threshold improved. An improvement of 1 bottle unit means that a patient could detect and/or recognize an odorant that was 10 times lower in concentration, when compared to baseline. For example, the recognition threshold (RT) of AA improved by 8 bottle units, also equivalent to an improvement of 8 orders of magnitude. In this case, the patient could detect a concentration of AA that was 10⁸ lower in concentration. Bottle unit numbers that are 5 or lower are considered to be in the range of normal.

TABLE 1

| | | DT | DT | DT | DT | RT | RT | RT | RT |
|---|---|---|---|---|---|---|---|---|---|
| | odor | Pyr | NO2B | Thio | AA | Pyr | NO2B | Thio | AA |
| Patient | Pre | 11 | 11 | 10 | 11 | 11 | 12 | 10 | 12 |
| Patient | 3 months | 7 | 9 | 4 | 4 | 7 | 9 | 4 | 4 |

DT = Detection Threshold;
RT = Recognition Threshold

### Example 6

A patient diagnosed or previously diagnosed with chemosensory dysfunction is treated with an intranasally administered stent comprising theophylline. The stent is administered to a nasal cavity of the patient. The stent comprises 2 layers: a poly(DL-lactide-co-glycolide) and poly(L-lactide) layer which comprises theophylline and a PLGA layer which comprises theophylline. The layers are configured to release theophylline at a dosage of about 15 μg to about 300 μg per day for about 90 days.

Administration of the stent produces an increase in the patient's recognition threshold and detection threshold within about 60 days. Further, this increase produces a significant increase in the patient's ability to taste, thus at least partially ameliorating the chemosensory dysfunction.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of treating a chemosensory dysfunction in a subject the method comprising: delivering a stent to a nasal or sinus cavity to treat the chemosensory dysfunction in the subject, wherein the nasal or sinus cavity comprises an inferior meatus, a middle meatus, a superior meatus, a sphenoethmoid recess, an olfactory cleft, an ethmoid sinus cavity, a maxillary sinus cavity, a frontal sinus cavity, or a sphenoid sinus cavity, wherein the stent:
(a) is at least partially impregnated with a phosphodiesterase (PDE) inhibitor or a salt thereof and is configured to release the PDE inhibitor or the salt thereof after delivering the stent to the nasal or the sinus cavity of the subject;
(b) comprises at least one coating layer on at least a portion of the stent, wherein the coating layer comprises the PDE inhibitor or the salt thereof and the coating layer is configured to release the PDE inhibitor or the salt thereof after delivering the stent to the nasal or the sinus cavity of the subject; or
(c) both (a) and (b),
and wherein the PDE inhibitor or the salt thereof comprises theophylline or a salt thereof, cilostazol or a salt thereof, or roflumilast or a salt thereof, or any combination thereof.

2. The method of claim 1, wherein the stent is placed within the nasal cavity, within a sinus cavity, or at a sinus ostium.

3. The method of claim 1, wherein the stent comprises the PDE inhibitor or the salt thereof which comprises theophylline or a salt thereof.

4. (canceled)

5. The method of claim 1, wherein the stent comprises the coating layer, and wherein the coating layer further comprises a release rate modifier.

6. (canceled)

7. The method of claim 5, wherein the release rate modifier comprises a bovine serum albumin, a human serum albumin, a collagen, a gelatin, a polylactic acid (PLA), a polyglycolide (PGA), a polylactic-co-glycolic acid (PLGA), a poly(lactide-co-glycolide), a poly(L-lactide-co-ε-ca-prolactone), a polyhydroxybutyrate (PHB), a polyhydroxyvalerate (PHV), a polydioxanone, a polyglactin, a polycaprolactone, a polyglyconate, a poly(glycolide-co-trimethylene carbonate), a poly(sebacic acid), a poly(ester urethane), a poly(ester urethane) urea, polyethylene glycol (PEG), or any combination thereof.

8. The method of claim 1, wherein the stent comprises the at least one coating layer, and wherein the stent further comprises a second coating layer.

9. The method of claim 8, further comprising a plurality of three or more coating layers.

10. The method of claim 1, wherein the stent is a bioabsorbable stent.

11. The method of claim 1, wherein the stent is a non-bioabsorbable stent.

12. The method of claim 1, wherein the stent comprises a PHB, a PHV, a polycaprolactone, or any combination thereof.

13. The method of claim 1, wherein the stent is configured to release the PDE inhibitor or the salt thereof over a period of time of from about 5 days to about 180 days.

14. The method of claim 1, wherein the stent is configured to release about 500 μg of the PDE inhibitor or the salt thereof per day.

15. The method of claim 1, wherein the chemosensory dysfunction is ageusia, hypogeusia, dysgeusia, parosmia, phantosmia, anosmia, hyposmia, dysosmia, smell loss, taste loss, or any combination thereof.

16.-17. (canceled)

18. The method of claim 1, wherein the subject before treatment has a decreased level of a cyclic nucleotide, a decreased level of a sonic hedgehog, or both, in a nasal mucus sample from the subject in comparison to a reference level associated with normal chemosensory function.

19. The method of claim 1, further comprising administering a second therapeutic.

20. The method of claim 19, wherein the second therapeutic is administered concurrently or consecutively.

21.-22. (canceled)

23. An implantable stent comprising: a phosphodiesterase (PDE) inhibitor or a salt thereof and/or a coating layer on at least a portion of the stent comprising the PDE inhibitor or the salt thereof, wherein the PDE inhibitor or the salt thereof is:
(a) impregnated in at least a portion of the stent;
(b) comprised in a coating layer on at least a portion of the stent; or
(c) both (a) and (b), wherein the stent is configured to release the PDE inhibitor or the salt thereof in a nasal cavity of a subject and wherein the PDE inhibitor or the salt thereof comprises theophylline, a salt thereof, cilostazol, a salt thereof, or any combination thereof.

24. A method of treating a chemosensory dysfunction in a subject the method comprising: introducing an implantable device to a nose to treat the chemosensory dysfunction in the subject, wherein the implantable device:
(a) is at least partially impregnated with a phosphodiesterase (PDE) inhibitor or a salt thereof;
(b) comprises at least one coating layer on at least a portion of the implantable device, wherein the coating layer comprises the PDE inhibitor or the salt thereof, or
(c) both (a) and (b),
wherein the implantable device is configured to release the PDE inhibitor or the salt thereof, and wherein the PDE inhibitor or the salt thereof comprises theophylline, a salt thereof, cilostazol, a salt thereof or any combination thereof.

25.-28. (canceled)

29. A kit comprising the implantable stent of claim 23 and a container.

30. A method of making the implantable stent of claim 23.
